(19) United States
(12) Patent Application Publication
(10) Pub. No.: US 2012/0004624 A1
(43) Pub. Date: Jan. 5, 2012

(54) ENHANCED ANTI-FLOW PROTECTION FOR AN INTRAVENOUS SET

(75) Inventors: Houston Brown, San Diego, CA (US); Rob Pecsar, Del Mar, CA (US)

(73) Assignee: CareFusion 303, Inc., San Diego, CA (US)

(21) Appl. No.: 12/828,048

(22) Filed: Jun. 30, 2010

(51) Int. Cl. A61M 39/28

(52) U.S. Cl. 604/250

(57) ABSTRACT

A device for holding an IV tube in a pumping apparatus and for selectively occluding the tube when the door of the pumping apparatus is opened, to prevent free flow of fluid in the tube. A flange provided on a sliding clamp is designed to prevent inadvertent finger slippage causing pushing of a tab positioned in the sliding direction from the flange, resulting in undesirable fluid leakage. The flange is made slip-resistant using a textured surface, having a concave shape, or having an angled top portion to prevent finger slippage or having a concave setback to guide a finger when pushing the sliding clamp.

### FIELD

The present disclosure is related to the field of intravenous (IV) infusion devices such as peristaltic pumps and the associated flexible IV tubing and other related devices. Specifically, the present disclosure relates to arrangements and methods for preventing free flow in an IV tube when the infusion pump is disengaged from the IV tube.

### BACKGROUND

It is a common practice to deliver fluids such as medications to a patient intravenously by means of a pumping device, such as a peristaltic pump, a four-finger pump, a diaphragm pump or a constant displacement pump. Such pumps are useful because they can deliver the medication in a highly controlled and precise fashion, and because they do so without coming in contact with the medication. The fluid is moved through a flexible IV tube by pressing a pumping member against the tube sufficiently to move the fluid in the tube downstream towards the patient. In the case of peristaltic mechanisms having multiple pumping fingers, the fingers are moved against the tube in a sequential manner from upstream to downstream to sequentially occlude the tube thereby moving the fluid in the tube downstream towards the patient. When the IV tube is mounted correctly in the pump, the IV tube is at all times occluded by one of the pumping members, thereby preventing the “free flow” of fluid to the patient. “Free flow” of medical fluids is undesirable since the flow of the fluid is uncontrolled and the prescribed treatment is not followed.

It is common for the peristaltic pumping mechanism to be located in a housing with a hinged door. The tube through which the fluid is to be moved is placed in contact with the pumping mechanism inside the door, with the ends of the tubing typically extending out the top and bottom of the door opening. As the door is shut over the tube, a platen on the inside of the door presses against the IV tube to provide a backing surface against which the pumping members can occlude the tube. The platen is typically spring loaded, although not always, against the pumping mechanism so that one or more of the pumping fingers of the pumping mechanism occludes the tube once the door is shut over the tube. The tube occlusion prevents free flow while the door is shut.

This arrangement of the IV tube relative to the pumping mechanism requires that there be some means for preventing flow in the tube when the door of the pump is open. Merely opening the pump door would relieve the tube from the occluding pumping mechanism/platen combination and free flow could possibly occur. This could result in the uncontrolled infusion of medication into the patient under the influence of the static head pressure in the tube, or blood from the patient could flow back into the IV tube. Known devices for preventing the unwanted flow in the tube include manual clamps on the tube separate from the infusion pump, and automatic occluding devices mounted on the pump. The manual devices require some manipulation skill on the part of the attending technician, and there is always the chance that the technician will forget to properly initiate the manual clamping process of the tube before the door of the pump is opened. Furthermore, the door may be accidentally opened, resulting in free flow in the tube.

Automatic devices mounted on the infusion pump for assisting in clamping and unclamping infusion tubes have improved. In particular, the reliability in timing the occlusion and release (unocclusion) of the tube with the disengagement and engagement, respectively, of the tube with the pumping members has improved. Typically, the action of opening the door is relied upon to initiate the occlusion of the IV tube by a clamp, and the action of closing the door is relied upon to initiate the release or unocclusion of the tube by an IV tube clamp. However, there still can occur the possibility of an inadvertent free flow of fluid through the IV tube due to operator error in regard to use of such devices. The above need has been partly addressed in the prior art by providing a mechanism called “flow stop.”

FIG. 1 is a perspective view of a prior art flow stop 300, disclosed in U.S. Pat. No. 5,453,098 to Botts et al. (“the '098 patent”), incorporated herein by reference. The flow stop 300 has a release tab 302 in the sliding direction (shown by arrow 111) of a slide clamp 304. The slide clamp 304 is shown fully withdrawn from the base 306. The release tab 302 is manually pressed in the sliding direction (arrow 111) as necessary to allow full insertion of the slide clamp 304 in the base 306. Pressing the release tab 302 results in allowing fluid to flow through an IV tube (not shown). The slide clamp 304 has a pushing surface 308 that has approximately the same height as the base 306. In operation, when seating the flow stop 300 in a pump, operators often find it convenient to push against the pushing surface 308 to firmly seat the flow stop 300 in a pump housing. However, because the pushing surface 308 is relatively narrow, there is a tendency for an operator's finger to inadvertently slip while pushing the slide clamp 304 in the sliding direction (arrow 111), for example, to properly seat the flow stop 300 in a pump. Because the release tab 302 is located in the sliding direction (arrow 111) from the pushing edge 308, when an operator pushes against the pushing edge 308 of the slide clamp 304, inadvertent slippage of an operator's finger from the pushing surface 308 may result in pressing the release tab 302, thereby causing undesired fluid leakage through the IV tube.

FIG. 2 is a perspective view of a prior art flow stop 320, disclosed in U.S. Pat. No. 7,303,175 to Smith et al. (“the '175 patent”) incorporated herein by reference. FIG. 2 shows the slide clamp 326 partially withdrawn from the base 328 of the flow stop 320. The slide clamp 320 is solves, in part, the above discussed problem of inadvertent pressing of the release tab. The configuration addresses the problem by placing the release tab 322 off-center in the sliding direction (arrow 111) with respect the flange 324 to avoid inadvertent activation. However, the off-center placement of the release tab 322 suffers from an operational shortcoming that because the flow stop 320 is subject to repetitive rotational force, the possibility of rotational wear and tear of the flow stop 320 increases.

In one aspect of the present disclosure, an improved anti-flow protection apparatus in needed.

### SUMMARY

The above-discussed and other needs for improved anti-flow protection devices and methods are disclosed.

In a first exemplary aspect, a mechanism for selectively preventing fluid flow through a resilient tube is disclosed. The mechanism comprises a base for holding the tube, a locking arm mounted on the base and having a moveable tab, a slide clamp, having a distal end and a proximate end, slidably mountable on the base for reciprocating movement in a sliding direction between an occluding position and an open position, the slide clamp having an aperture through which the tube extends when the tube is mounted within the mechanism, the aperture having a constricted region at which the tube is occluded when the tube is within the constricted region, and an expanded region at which the tube is not occluded when the tube is within the expanded region, and a flange at the distal end of the slide clamp and configured for interaction with an operator finger for pushing the slide clamp in a sliding direction toward the open position, the moveable tab being directly behind the flange in the sliding direction, the flange having a slip preventive feature preventing slippage of the operator finger from the flange at least in the sliding direction, wherein the locking arm locks the slide clamp in the occluding position until released by actuation of the moveable tab.

In a second exemplary aspect, A method of preventing fluid leakage from a tube during loading the tube in a pump, comprising the steps of passing the tube through an opening in a slide clamp, the slide clamp provided for occluding the tube when the slide clamp is in an occluding position and allowing passage of fluid through the tube when the slide clamp is in an open position, the slide clamp being slideable in a sliding direction between the occluding position and the open position, the slide clamp comprising a release tab in the sliding direction with respect to a flange coupled to the slide clamp, pushing, with an operator's finger, on the flange in the sliding direction to seat the slide clamp in the pump, and preventing inadvertent slippage of the operator's finger in the sliding direction with a slip prevention feature on the flange, wherein the release tab is configured to deactivate the slide clamp upon closing a door of the pump, thereby allowing passage of fluid through the tube.

In a third exemplary aspect, a slide clamp for use in a fluid pump is disclosed. The slide clamp comprises a flange positioned at a distal end of the slide clamp, the flange configured to reciprocate in a sliding direction. The flange comprises a height greater than a thickness of the slide clamp in a direction substantially perpendicular to the sliding direction and a slip preventive feature preventing slippage of an operator's finger from the flange at least in the sliding direction.

The foregoing and other features, aspects and advantages of the embodiments of the present disclosure will become more apparent from the following detailed description and accompanying drawings.

### BRIEF DESCRIPTION OF THE DRAWINGS

FIG. 1 is a perspective view of a prior art flow stop having a release tab in the sliding direction of a slide clamp, showing the slide clamp fully withdrawn from the base.

FIG. 2 is a perspective view of a prior art flow stop having a release tab in a direction other than the sliding direction from a slide clamp, showing the slide clamp partially withdrawn from the base.

FIG. 3 is a perspective view of the front of an infusion pump, in accordance with certain configurations of the present disclosure.

FIG. 4 is a perspective view of a flow stop showing a slide clamp fully withdrawn from the base, in accordance with certain configurations of the present disclosure.

FIG. 5 is a plan view of the top of the slide clamp shown in FIG. 4.

FIG. 6 is a section view of the slide clamp shown in FIG. 5, taken along the line III-III.

FIG. 7 is a section view of the base shown in FIG. 4, taken along the line IV-IV of FIG. 4.

FIG. 8 is a section view of the base shown in FIG. 4, taken along the line IV-IV of FIG. 4, depicting the release tab pressed against the tower.

FIG. 9 is a section view of the flow stop shown in FIG. 4, depicting the slide clamp locked in the occluding position.

FIG. 10 is a section view of the flow stop shown in FIG. 4, with the slide clamp in the open position.

FIG. 11 is a perspective view of a slide clamp, depicting a flange with textured surface, in accordance with embodiments of the present disclosure.

FIG. 12A is a perspective view of a slide clamp, depicting a concavely shaped flange, in accordance with certain configurations of the present disclosure.

FIG. 12B is a perspective view of a slide clamp, depicting a flange with a concave setback, in accordance with certain configurations of the present disclosure.

FIG. 13A is a perspective view of a slide clamp, depicting a flange with an angled top portion, in accordance with certain configurations of the present disclosure.

FIG. 13B is a perspective view of a slide clamp, depicting a flange with an angled top portion and an angled bottom portion, in accordance with certain configurations of the present disclosure.

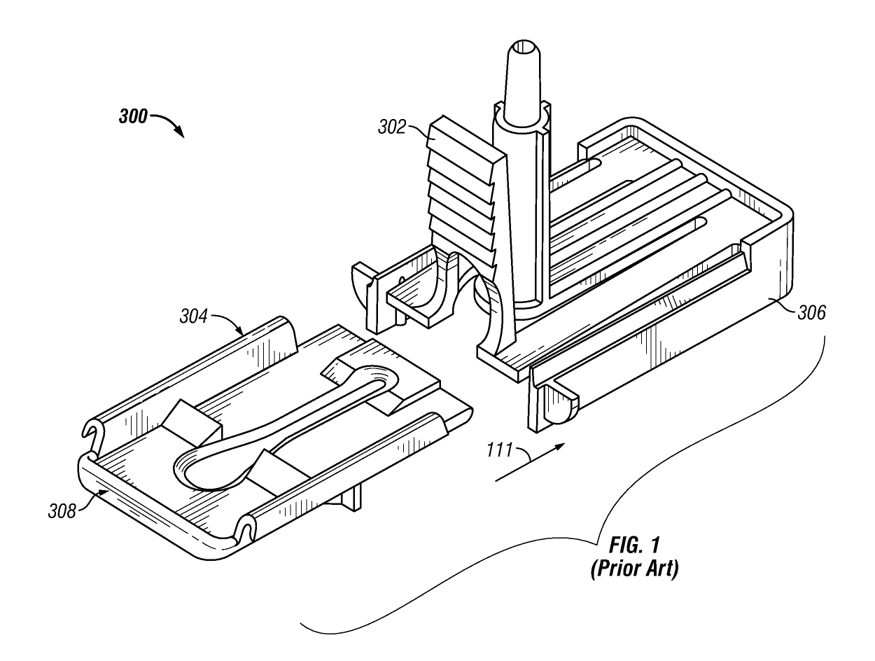

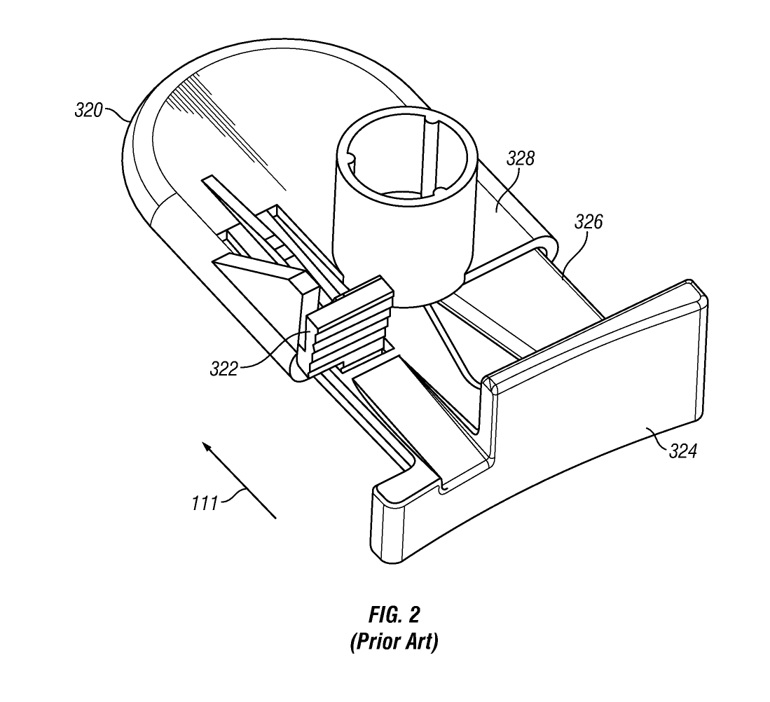

### DETAILED DESCRIPTION

Certain configurations of the present disclosure address and solve problems related to accidental fluid discharge when a pump door of a fluid delivery pump is open by provision of a flange fitted on a slide clamp that is a part of a flow stop mechanism. In certain aspects, the flange is configured to prevent accidental slippage of finger that may result in pressing of a release tab. Pressing the release tab when the pump door is open and a disposable portion is being loaded in the pump may defeat a flow-stop mechanism before the disposable portion is loaded completely, thereby causing undesirable fluid flow.

In certain configurations, the pump door is configured to deactivate the slide clamp upon closure of the pump door. Broadly speaking, at least two pump door configurations are possible. In the first pump door configuration, one or more bosses are included to control operation of the flow stop, resulting in deactivation of the flow stop after the pump door is closed. Additional operational details of such a pump door configuration can be found in the previously referenced U.S. Pat. No. 5,453,098. In the second pump door configuration, an opening is provided in the pump door through which a latching arm travels to deactivate the flow stop upon closure of the pump door. Additional operational details of such a pump door configuration can be found in U.S. Pat. No. 6,629,955 to Morris et al., incorporated herein by reference.

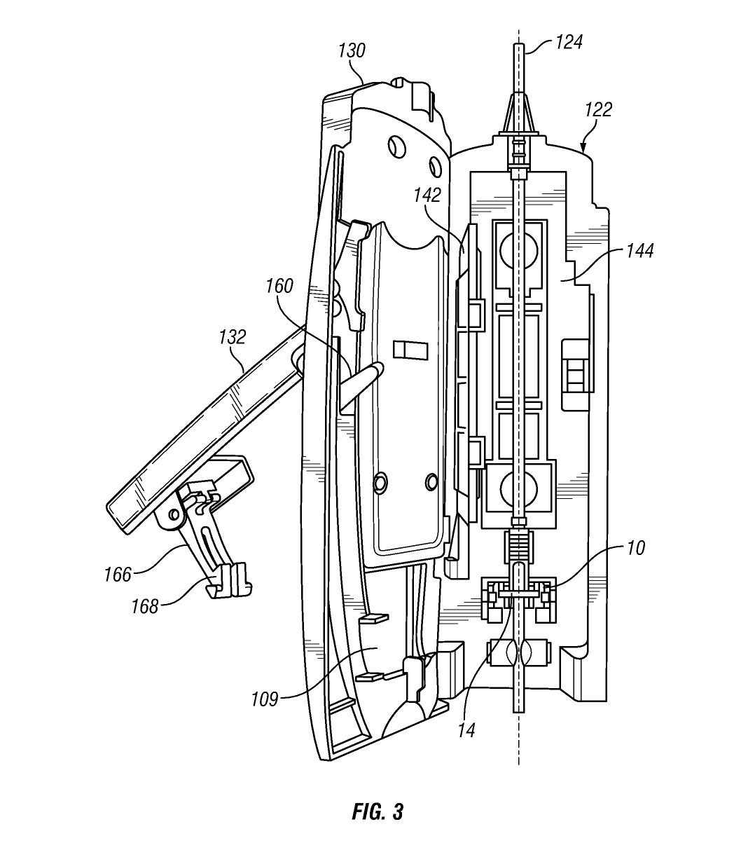

FIG. 3 shows an exemplary fluid pump having a pump door with a latching arm (second pump door configuration, as described above). Referring now to FIG. 3, a perspective view of a fluid delivery pump 122 is shown with the outer pump door 130 opened. A platen 142 is mounted between the door 130 and the pumping mechanism 144. The illustrated pumping mechanism 144 is of the “four finger” type. The operation of four finger pumps is well known to those skilled in the art and no further operational details are provided here. The IV tube 124 is mounted on the pumping mechanism 144. The handle 132 includes a latch arm 160 positioned to engage a yoke 162 located on the housing 164 of the pump 122. Engagement of the yoke 162 by the latch arm 160 will permit the door 130 to remain locked in the closed position. The handle 132 also includes a sear 166 having at least one hook 168, and in the illustrated embodiment, the sear 166 has two hooks. A flow stop 10, including a slide clamp 14, is configured to prevent inadvertent fluid discharge during operation of the pump 122. The door 130 has an opening 109 through which the hook 168 can couple with facilitates operation of the flow stop 10.

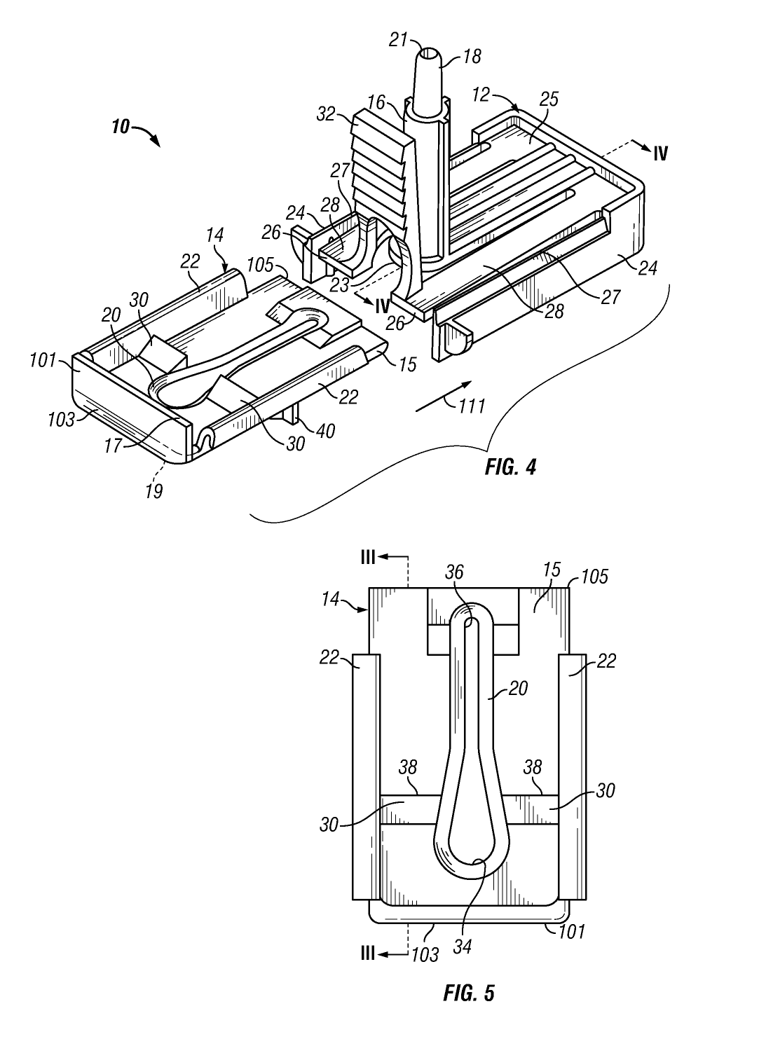

As shown in FIG. 4, the flow stop 10 consists generally of a relatively open, box shaped base 12 and a mating slide clamp 14. Both parts 12, 14 can be formed, for example, by injection molding from various plastic materials, although other manufacturing methods and materials may be used. The solid rectangular body 15 of the slide clamp 14 is shaped and sized to fit slidingly within the base 12. The slide clamp 14 has a proximate end 105 and a distal end 103, the distal end 103 being positioned farther away from the base 12 than the proximate end 105. The base 12 has a tower 16 formed on the top surface 25 of the base 12, with the tower 16 extending upwardly from the base 12, substantially perpendicular to the base 12. The top end 21 of the tower 16 is formed as a male tube connector 18 over which a pumping tube can be attached. The pumping tube can be attached by other means if desired. The open bottom end 23 of the tower 16 is attached to the base 12, and it is formed as a female tube connector into which an IV tube can be attached. The IV tube and the pumping tube can be the same tube if desired, simply passing through the tower 16.

The body 15 of the slide clamp 14 is penetrated from its top surface 17 to its bottom surface 19 by an elongated aperture 20. The elongated dimension of the aperture 20 is arranged on the slide clamp 14 to be parallel to the direction of the relative sliding movement between the base 12 and the slide clamp 14. Two side edges of the body 15 of the slide clamp 14 are fitted with rails 22 that lie parallel to the sliding direction (arrow 111). When the slide clamp 14 is slidingly engaged with the base 12, the rails 22 fit in a sliding fashion on two rail channels 27 on the top of the base 12 at the top of two frames 24 formed on the edges of the base 12. Alignment of the slide clamp 14 with the base 12 is accomplished by the fit of the rails 22 over the rail channels 27 of the frames 24, and by the fit of the body 15 of the slide clamp 14 between the frames 24.

Two flexible cantilevered locking arms 28 are molded into the top of the base 12, with their distal free ends 26 biased downwardly below the top surface 25 of the base 12. Biasing of the free ends 26 downwardly is accomplished by molding the locking arms 28 in a downwardly sloped configuration, but the biasing could also be accomplished by the use of springs or other means. A release tab 32 is formed on the locking arms 28, projecting upwardly from the locking arms 28 substantially parallel to the longitudinal axis of the tower 16. In the free state, when the locking arms 28 are sloped downwardly relative to the top surface 25 of the base 12, the release tab 32 is spaced away from the outer surface of the tower 16. The free ends 26 of the locking arms 28 can be flexed upwardly by pressing the release tab 32 toward the tower 16. Without departing from the spirit of the disclosure, one locking arm 28 can be used in place of the two shown, or each locking arm 28 can have a separate release tab 32.

Two locking projections 30 are molded on the top surface 17 of the body 15 of the slide clamp 14, with the projections 30 taking the form of ramps. The locking projections 30 are transversely positioned on the slide clamp 14 to align with the free ends 26 of the locking arms 28 when the slide clamp 14 is inserted into the base 12. The locking projections 30 are also longitudinally positioned to prevent the slide clamp 14 from being inserted into the base 12 far enough to move from its occluding position to its open position.

A flange 101 is provided at the distal end 103 of slide clamp 14, the distal end 103 being located farther away from the sliding direction (arrow 111) with respect to the proximate end 105 and the release tab 32. The flange 101 is provided on the slide clamp 14 to facilitate movement of the slide clamp 14 to the open position by pushing against the flange 101 in the sliding direction (arrow 111). The flange 101 is configured to be slip-resistant (i.e., prevent an inadvertent finger slip) when pushing the flange in the sliding direction. In certain configurations of the present disclosure, the flange 101 has a height great enough to allow secure placement of an operator's finger during pushing operation. To prevent finger-slip, the height of the flange 101, in the direction perpendicular to the sliding direction (arrow 111) may be chosen to be 2 millimeters or more, generally in the 5-10 millimeters range. In certain embodiments, the height of the flange 101 in the direction substantially perpendicular to the slide clamp 14 is larger than thickness of the slide clamp 14, thereby providing better support for an operator's finger during pushing operation than the slide clamp 14. The height of the flange 101 is practically limited in the depicted embodiment by the opening 109 in the pump door 130 so that the flange 101 does not inhibit closing of the pump door 130 during fluid delivery operation. However, in other embodiments, when a pump door 130 opening is not a limiting factor, the height of the flange 101 may be made greater to provide further assurance of finger slippage prevention.

As seen in FIG. 5, the elongated aperture 20 through the slide clamp 14 has an open end 34 shaped essentially as a round hole with a sufficiently large diameter to allow a selected IV tube to pass through the open end 34 without being occluded. Preferably, the diameter of the open end 34 is large enough to allow the IV tube to remain unrestricted. The other end of the aperture 20 is a relatively narrow slot 36. The width of the slot 36 is sufficiently small that a selected IV tube passing through the slot 36 would be completely occluded and would remain occluded against a foreseeable range of fluid pressures in the IV tube. The range of pressures against which the IV tube would remain occluded would include at least the static head anticipated during normal use of the infusion apparatus.

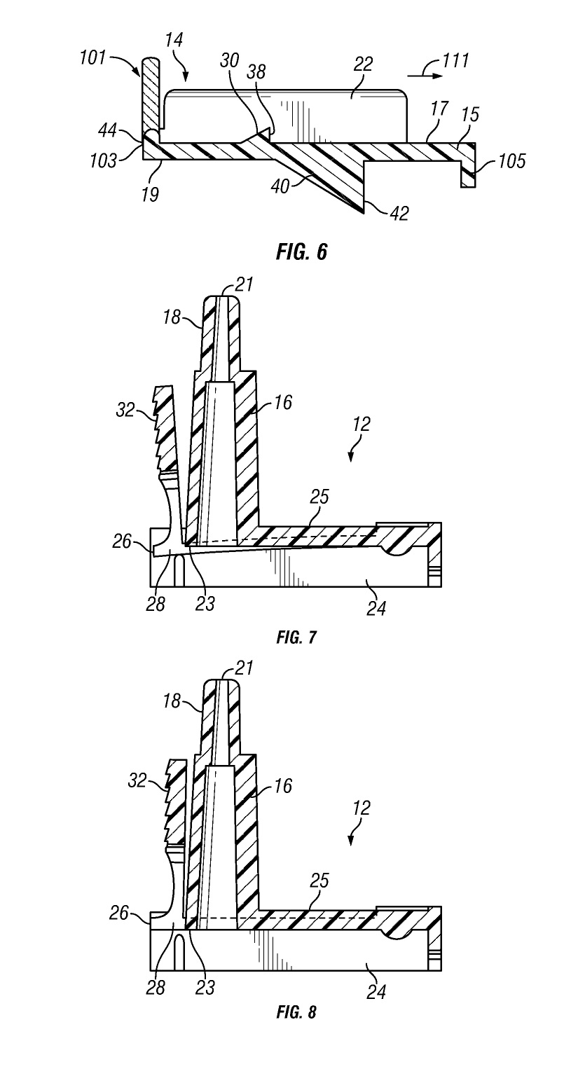

As seen in FIG. 6, the locking projections 30 project upwardly from the top surface 17 of the body 15 of the slide clamp 14, presenting a substantially vertical locking face 38 to engage the free ends 26 of the locking arms 28, when the slide clamp 14 is in its occluding position. One or more pulling projections 40 project downwardly from the bottom surface 19 of the body 15. Each of the pulling projections 40 presents a substantially vertical pulling face 42 which will interact with the latch (132, FIG. 3) on the door of the pump housing (122, FIG. 3) to pull the slide clamp 14 partially out of engagement with the base 12 before the door is opened. Pulling the slide clamp 14 partially out of the base 12 moves the slide clamp 14 from its open position to its occluding position. The body 15 of the slide clamp 14 also presents the flange 101 on the distal end 103, against which the door 130 of the housing pushes to fully insert the slide clamp 14 into the base 12, after the door 130 is closed. Pushing the slide clamp 14 in the sliding direction (arrow 111) for full insertion into the base 12 moves the slide clamp 14 from its occluding position to its open position.

Still referring to FIG. 6, the flange 101 is shown. In the illustrated embodiment, the flange 101 is taller than the rails 22, the increased height compared to the prior art facilitating a slip-resistant contact between an operator's finger and the slide clamp 14, when the slide clamp 14 is being pushed in the sliding direction (arrow 111).

FIG. 7 illustrates the downward slope of the locking arms 28, which is molded into the locking arms 28 to create the downward bias to engage the free ends 26 of the locking arms 28 with the locking faces 38 on the locking projections 30. The separation between the release tab 32 and the side of the tower 16 can also be seen, as it exists when the locking arms are unrestrained. FIG. 8 shows the upwardly flexed position of the free ends 26 of the locking arms 28 which results from pressing the release tab 32 toward the tower 16. In this view, the release tab 32 is shown contacting the tower 16, but it should be understood that the free ends 26 can be flexed upwardly a sufficient amount to release the locking arms 28 from the locking projections 30, without actually causing the release tab 32 to contact the tower 16.

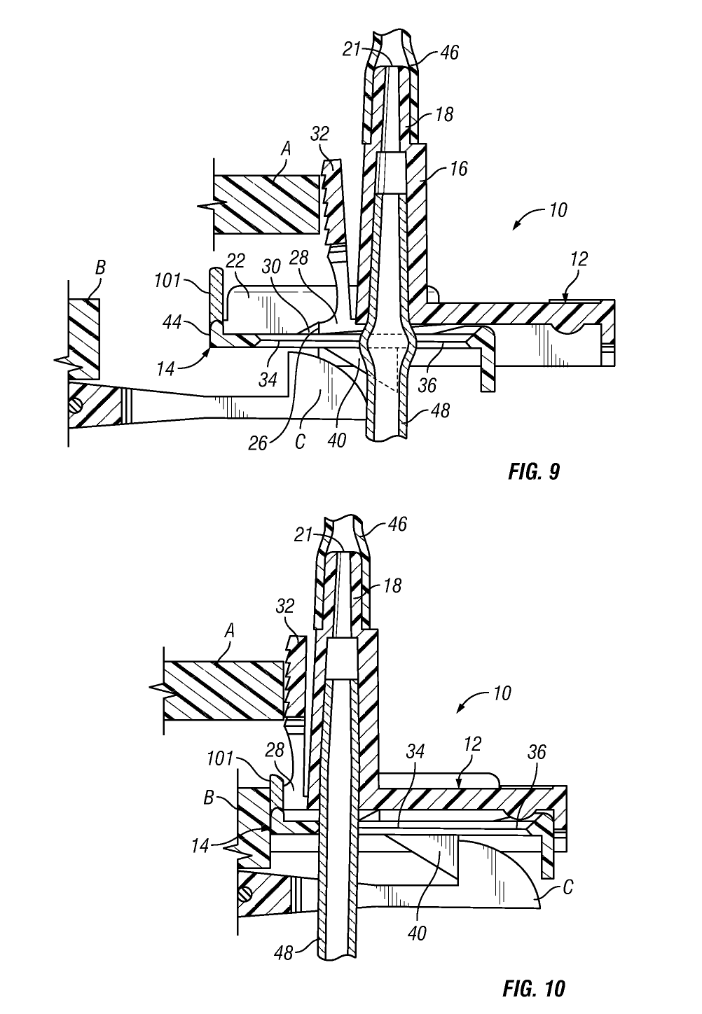

FIGS. 9 and 10 show in general how embodiments of the flow stop 10 of the present disclosure interact with a pump door having one or more bosses included to control operation of the flow stop (the first pump door configuration, as described above). Operational details of interaction between the flow stop 10 and a pump door with a latching arm (the second pump door configuration, as described above) are consistent with the description in the previously referenced U.S. Pat. No. 6,629,955 and have been omitted for the sake of brevity. FIG. 9 shows the slide clamp 14 in its occluding position relative to the base 12, with the slide clamp 14 partially withdrawn from the base 12 and the free ends 26 of the locking arms 28 engaging the locking projections 30 to hold the slide clamp 14 in its occluding position. This position of the slide clamp 14 is achieved before the door (e.g., 130, FIG. 3) is opened and maintained until after the door 130 is closed. FIG. 10 shows the slide clamp 14 in its open position, with the slide clamp 14 fully inserted within the base 12 and the free ends 26 of the locking arms 28 flexed upwardly a sufficient amount to clear the locking projections 30.

Operative elements of the door and latch mechanism are shown schematically and designated as elements A, B, and C, to illustrate their interaction with the flow stop 10 of the present disclosure. The releasing boss A is formed on the door and positioned to contact the release tab 32 as the door is moved to the shut position, and to press the release tab 32 toward the tower 16. A pushing boss B is formed on the latch mechanism and positioned to contact the flange 101 on the slide clamp 14, as the latch is engaged, to push the slide clamp 14 from its occluding position to its open position, Finally, one or more pulling hooks C are formed on the latch mechanism and positioned to contact the pulling projections 40 as the latch is disengaged to pull the slide clamp 14 from its open position to its occluding position.

Element A of the door moves generally to the right as seen in FIGS. 9 and 10 when the door is moved to the shut position. Elements B and C of the latch mechanism move generally to the left as seen in FIGS. 9 and 10 when the latch is being disengaged, and to the right when the latch is being engaged, it being understood that other elements (not shown) of the latch mechanism perform the actual latching of the door 130 in the shut position. In addition, pulling hooks C can rotate in the clockwise direction from the position shown, relative to the remainder of the latch mechanism, against a spring bias.

To use the flow stop 10 of the present disclosure, the slide clamp 14 is fully inserted into the base 12 until the open end 34 of the aperture 20 aligns with the longitudinal axis of the tower 16. The release tab 32 is manually pressed toward the tower 16 as necessary to allow full insertion of the slide clamp 14. A pumping tube 46 is selected for its appropriate size, flexibility, and durability. One end of the selected pumping tube 46 is attached to the tube connector 18 at the top end 21 of the tower 16 by being fit over the tube connector 18. One end of a selected IV tube 48 is threaded through the open end 34 of the aperture 20 and attached to the base 12 by being fit into the bottom end 23 of the tower 16. The slide clamp 14 is then manually withdrawn from the base 12 to occlude the IV tube 48, as depicted in FIG. 9.

The other end of the pumping tube 46 is connected to a supply (not shown) of the chosen fluid to be pumped. The door 130 of the pump housing 122 is opened and the flow stop 10 is placed inside the door 130 with the base 12 securely mounted to the pump housing and the free ends 26 of the locking arms 28 projecting outwardly from the pump housing. The pumping tube 46 is placed in contact with the pumping mechanism (e.g., 144, FIG. 3), and the door 130 is shut. FIG. 9 shows the slide clamp 14 in its occluding position, with the release boss A about to contact the release tab 32 as the door 130 is shut. As the door 130 is completely shut, the release boss A presses the release tab 32 toward the tower 16, flexing the locking arms 28 upwardly. After the door 130 is shut, the latching mechanism is latched, causing the pushing boss B to push the slide clamp 14 to its open position. FIG. 10 shows the slide clamp 14 in its open position, with the pulling hooks C having pivoted behind the pulling projections. The pump can then be operated in the conventional fashion to purge the IV tube 48 of air, and the IV tube 48 can be connected to a venous access site.

If the pump door 130 is to be opened, the latching mechanism must first be disengaged, which will move the elements B and C to the left, causing the pulling hooks C to contact the pulling projections 40 and pull the slide clamp 14 to the left, to its occluding position. The latching mechanism can be constructed by known means so that only after this occlusion occurs will the door 130 be unlatched. At this time, the door 130 can be opened. The locking arms 28, having engaged the locking projections 30, maintain the slide clamp 14 in its occluding position, even if the slide clamp 14 is pushed toward the base 12 with considerable force.

The latching mechanism can also be constructed by known means so that when the door 130 has been unlatched, the latching mechanism cannot be moved back to its latched position until the door 130 has been shut. Therefore, if the door 130 is to be shut, the release boss A will press the release tab 32 toward the tower 16 to release the locking arms 28 from engagement with the locking projections 30, and the slide clamp 14 can be subsequently moved to the open position as explained before.

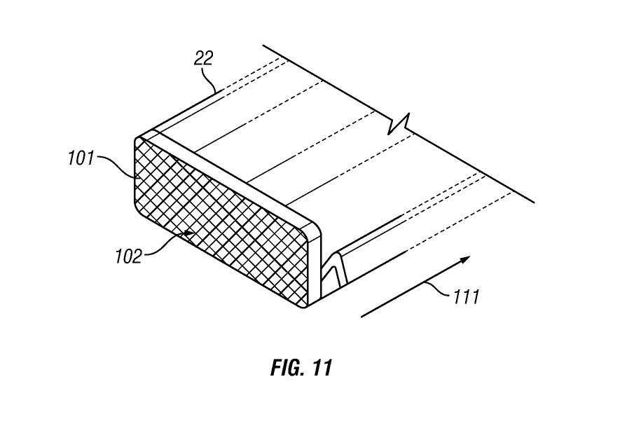

FIG. 11 is a perspective view of a slide clamp 22, showing one embodiment of the flange 101 with textured surface 102, in accordance with embodiments of the present disclosure. Other details of the slide clamp are as shown in FIGS. 4-10, and are omitted in FIG. 11 to selectively highlight certain aspects of the present disclosure. The textured surface 102 helps prevent accidental sliding of an operator's finger in the process of moving the slide clamp 22 in the sliding direction, shown as arrow 111. One skilled in the art will appreciate that a variety of surfaces are possible to provide slip-resistant texture to the textured surface 102, including grooves, protrusions or cross-hatched patterns, etc. Further, the textured surface may include an applied surface, such as a “gripping” surface made of sponge rubber, for example.

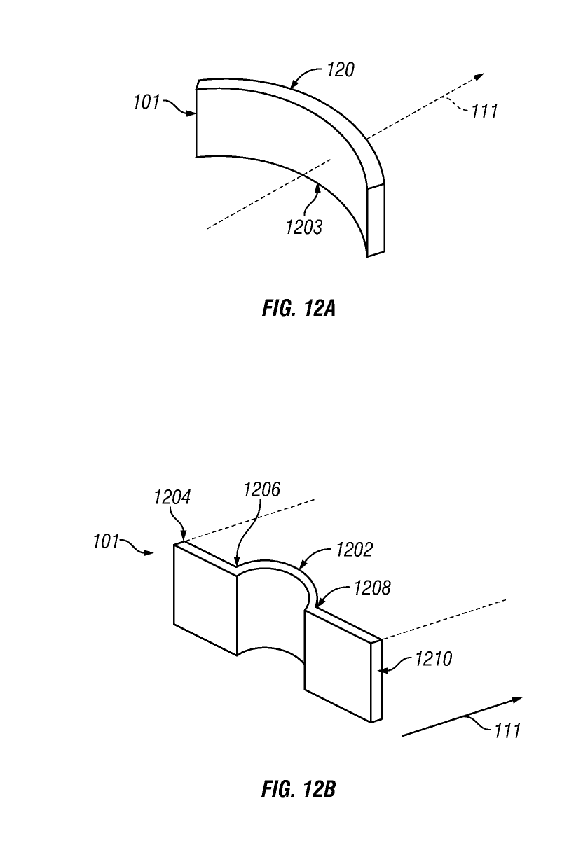

FIG. 12A is a perspective view of the flange 101 in accordance with certain configurations of the present disclosure. The flange 101 is configured to have a concave shape protruding in the direction of the sliding movement, shown by arrow 111. The concavity may be achieved by contouring the upper edge 1201 and the lower edge 1203 of the flange 101 to have an elliptical, a semicircular, a rectangular, or any other geometric shape. The concave shape of the flange 101 may help securely position an operator's finger, thereby preventing inadvertent slippage.

FIG. 12B is a perspective view of the flange 101 in accordance with certain configurations of the present disclosure. The flange 101 is configured to have a concave setback 1202 in the sliding direction, shown by arrow 111, extending from edge 1206 to edge 1208. In general, the edges 1206, 1208 are positioned inside the edges 1204 and 1210 of the flange 101. In certain configurations, the concave setback 1202 is configured to have a generally semicircular, ellipsoid or rectangular shape. In one aspect, the concave setback 1202 may act as a tactile guide for an operator's finger, thereby preventing inadvertent slippage in the sliding direction 111, during operation.

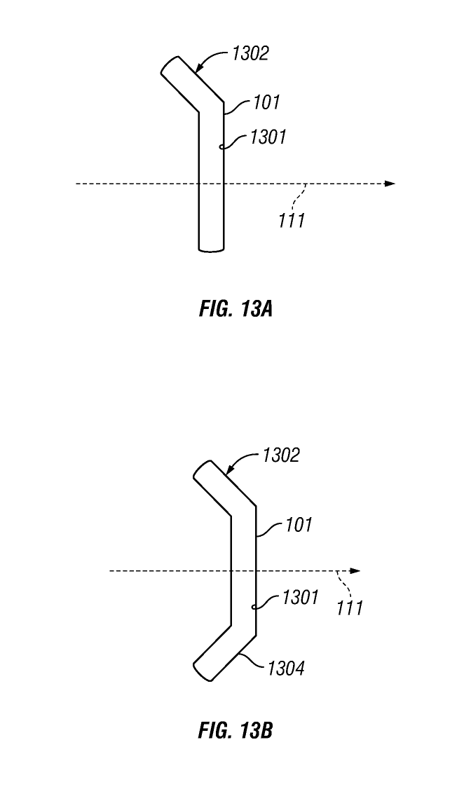

FIG. 13A is a side view of a flange 101, depicting an angled top portion (“top lip”) 1302. When an operator's finger pushes against the flange 101 to move in the direction of arrow 111, the angled portion 1302 may prevent inadvertent upwardly slippage of the finger, thereby reducing chances of inadvertent pressing of the release tab 32 (not shown in FIG. 13A). In relation to the vertical portion 1301 of the flange 101, the top portion 1302 may be configured to be at an angle (as shown in FIG. 13A), or to be curved or have any other suitable design to prevent inadvertent finger slippage.

FIG. 13B is a side view of a flange 101, showing an angled top portion 1302 and an angled bottom portion (“bottom lip”) 1304. When an operator's finger pushes against the flange 101 to move in the direction of arrow 111, the well formed by the vertical portion 1302 and the angled portions 1302, 1304 prevents inadvertent upward or downward slippage of the finger, thereby reducing chances of inadvertent pressing of the release tab 32 (not shown in FIG. 13B). In relation to the vertical portion 1301 of the flange 101, the top portion 1302 and the bottom portion 1304 may be configured to be at an angle (as shown in FIG. 13B), or to be curved or have any other suitable design to prevent inadvertent finger slippage.

In certain configurations, the flange 101 comprises more than one of the various slip-resistant designs. For example, the flange 101 is designed to have an angled top portion and at the same time be concavely shaped. Furthermore, although in the illustrated configurations, the flange 101 is shown to be generally rectangular, several other shapes (e.g., oval, circular) are possible within the scope of the present disclosure. In certain configurations, the flange 101 is fabricated as a separate element that is attached to the flow stop using rivets or glue, etc. In certain configurations, the flange 101 and the flow stop 10 form a single piece, typically manufactured by injection molding. Furthermore, the flange 101 comprises variety of materials, such as slip-resistant rubber, plastic, etc.

Although embodiments of the present disclosure have been described and illustrated in detail, it is to be clearly understood that the same is by way of illustration and example only and is not to be taken by way of limitation, the scope of the present invention being limited only by the terms of the appended claims.

What is claimed is:

1. A mechanism for selectively preventing fluid flow through a resilient tube, comprising:
a base for holding the tube;
a locking arm mounted on the base and having a moveable tab;
a slide clamp, having a distal end and a proximate end, slidably mountable on the base for reciprocating movement in a sliding direction between an occluding position and an open position, the slide clamp having an aperture through which the tube extends when the tube is mounted within the mechanism, the aperture having a constricted region at which the tube is occluded when the tube is within the constricted region, and an expanded region at which the tube is not occluded when the tube is within the expanded region;
a flange at the distal end of the slide clamp and configured for interaction with an operator finger for pushing the slide clamp in a sliding direction toward the open position, the moveable tab being directly behind the flange in the sliding direction, the flange having a slip preventive feature preventing slippage of the operator finger from the flange at least in the sliding direction;
wherein the locking arm locks the slide clamp in the occluding position until released by actuation of the moveable tab.

2. The mechanism of claim 1, wherein the slip prevention feature comprises a flange having a height greater than a thickness of the slide clamp.

3. The mechanism of claim 1, wherein the flange comprises a textured surface.

4. The mechanism of claim 1, wherein the flange is configured to have a concave shape.

5. The mechanism of claim 1, wherein the flange comprises a concave setback.

6. The mechanism of claim 1, wherein the flange comprises an angled top portion to prevent inadvertent slippage of the operator finger in an upward direction.

7. The mechanism of claim 6, wherein the flange further comprises an angled bottom portion to prevent inadvertent slippage of the operator finger in a downward direction.

9. A method of preventing fluid leakage from a tube during loading the tube in a pump, comprising the steps of:
passing the tube through an opening in a slide clamp, the slide clamp provided for occluding the tube when the slide clamp is in an occluding position and allowing passage of fluid through the tube when the slide clamp is in an open position, the slide clamp being slideable in a sliding direction between the occluding position and the open position, the slide clamp comprising a release tab in the sliding direction with respect to a flange coupled to the slide clamp;
pushing, with an operator's finger, on the flange in the sliding direction to seat the slide clamp in the pump; and
preventing inadvertent slippage of the operator's finger in the sliding direction with a slip prevention feature on the flange;
wherein the release tab is configured to deactivate the slide clamp upon closing a door of the pump, thereby allowing passage of fluid through the tube.

10. The method of claim 9, wherein the slip prevention feature comprises a flange having a height greater than a thickness of the slide clamp.

11. The method of claim 9, wherein the slip prevention feature comprises a textured surface.

12. The method of claim 9, wherein the flange is configured to have a concave shape.

13. The method of claim 9, wherein the flange comprises a concave setback.

14. The method of claim 1, wherein the slip prevention feature comprises an angled top portion to prevent inadvertent slippage of the operator finger in an upward direction.

15. The method of claim 14, wherein the slip prevention feature further comprises an angled bottom portion to prevent inadvertent slippage of the operator finger in a downward direction.

16. A slide clamp for use in a fluid pump, the slide clamp comprising a flange positioned at a distal end of the slide clamp, the flange configured to reciprocate in a sliding direction, the flange comprising:
a height greater than a thickness of the slide clamp in a direction substantially perpendicular to the sliding direction; and
a slip preventive feature preventing slippage of an operator's finger from the flange at least in the sliding direction.

17. The slide clamp of claim 16, wherein the slip prevention feature comprises a textured surface.

18. The slide clamp of claim 16, wherein the slip prevention feature comprises a concave shape.

19. The slide clamp of claim 16, wherein the slip prevention feature comprises a concave setback.

20. The slide clamp of claim 16, wherein the slip prevention feature comprises an angled top portion to prevent inadvertent slippage of the operator finger in an upward direction.

21. The slide clamp of claim 20, wherein the slip prevention feature comprises an angled bottom portion to prevent inadvertent slippage of the operator finger in a downward direction.
